(19) United States
(12) Patent Application Publication
(10) Pub. No.: US 2025/0090824 A1
(43) Pub. Date: Mar. 20, 2025

(54) MICRONEEDLE THERAPY DEVICE AND SYSTEM, AND METHOD FOR INJECTING ACTIVE INGREDIENTS INTO SKIN USING SAME

(71) Applicant: CURIOSIS CO., LTD, Seoul (KR)

(72) Inventors: Ho Young YUN, Seoul (KR); Hee Don AHN, Seoul (KR); Ki Chan KIM, Seoul (KR); Ji Hoon SHIN, Seoul (KR); Kyun Tae KIM, Seoul (KR)

(73) Assignee: CURIOSIS CO., LTD, Seoul (KR)

(21) Appl. No.: 18/950,550

(22) Filed: Nov. 18, 2024

Related U.S. Application Data
(63) Continuation of application No. PCT/KR2023/006821, filed on May 19, 2023.

(30) Foreign Application Priority Data
May 20, 2022 (KR) 10-2022-0062059
Mar. 22, 2023 (KR) 10-2023-0037330

(51) Int. Cl. A61M 37/00

(52) U.S. Cl. CPC A61M 37/0015; A61M 2037/0023; A61M 2205/33; A61M 2210/04

(57) ABSTRACT

A microneedle therapy device equipped with a needle cover according to an example embodiment of the present disclosure includes a pneumatic pump, a valve configured to switch a supply and cutoff of a compressed air from the pneumatic pump into the needle cover, a microneedle arranged inside the needle cover and configured to move forward and backward from an opening of the needle cover, and a controller configured to control an opening and closing of the valve and the movement of the microneedle. The controller is configured to control an opening time of the valve based on information input by a user.

### CROSS-REFERENCE TO RELATED APPLICATIONS

This application is a continuation of International Application No. PCT/KR2023/006821 filed on May 19, 2023, which claims priority to Korean Patent Application No. 10-2022-0062059 filed on May 20, 2022 and Korean Patent Application No. 10-2023-0037330 filed on Mar. 22, 2023, the entire contents of which are herein incorporated by reference.

### TECHNICAL FIELD

The present disclosure relates to a microneedle therapy device, a microneedle therapy system, and a method for injecting active ingredients into a skin using the same, and more particularly, to a microneedle therapy device and a microneedle therapy system which have an improved configuration in which fine holes are formed in the skin with microneedles, and then the active ingredients such as pharmacological agents are injected into the skin through the fine holes to induce production of collagens by stimulation to fibroblasts, negative and positive pressures are alternately generated, compressed air is supplied as pneumatic pressure to directly push the active ingredients so that the active ingredients are easily injected into the skin, thereby alleviating pain, achieving good active ingredient injection capability, improving a skin regenerative therapy and skin regeneration efficacy.

### BACKGROUND

In general, a health condition of a skin has a large impact on the appearance. Thus, in recent years, various methods have been developed for the purpose of lightening the skin, improving wrinkles, moisturizing the skin, increasing resilience of the skin, and the like, in addition to the purpose of treating a skin disease.

As is well known, the human skin may be roughly classified into an epidermal layer (epidermis), a dermal layer (dermis) and a fat layer.

Among these, the dermal layer occupying a substantial portion of the skin is composed of a papillary layer and a plexiform layer. Capillaries, lymphatic tubes and the like are located in the papillary layer. Collagens as collagen fibers associated with wrinkles in the skin, elastin as elastic fibers that give elasticity to the skin, a substrate and the like are contained in the plexiform layer.

A visual condition of the skin is greatly dependent on a health status of the dermal layer. Accordingly, various methods implemented for improving the condition of the skin are often targeted for the dermal layer.

Since the dermal layer is protected by the epidermal layer as described above, when pharmacological agents to be delivered to the dermal layer are applied onto the skin, an amount of the pharmacological agents which reach the dermal layer and a reaching speed thereof may be drastically reduced.

Accordingly, in order to rapidly deliver the pharmacological agents applied onto the epidermal layer to the dermal layer, a method of applying pressure or ultrasonic vibrations has been proposed. This method has the advantage of not damaging the epidermal layer but requires a part for generating the pressure or the ultrasonic vibrations. This may cause an increase in cost and size.

As an approach for solving such matters, a wound treatment efficacy using fine needles has been disclosed in 1995, and a method (microneedle therapy system “MTS”) for forming passages in the epidermal layer with microneedles and applying pharmacological agents, which induce a desired efficacy, to the skin so that they are delivered to the dermal layer, has been widely used.

The MTS uses the microneedles having a thickness of 0.2 millimeter (mm) or less and a length of 2 mm or less to physically damage the epidermal layer and the dermal layer. This stimulates fibroblasts to induce production of collagens. Further, the MTS injects pharmacological agents such as vitamins, hyaluronic acids or the like to be sufficiently adsorbed to the epidermal layer and the dermal layer, thereby improving a treatment efficacy. As a technology for improving such a treatment efficacy, a number of documents including the following documents in the related art have been disclosed.

However, in a technology of using the microneedles, the pharmacological agents may not be smoothly injected to sites from which the microneedles are withdrawn. This may result in poor treatment and regeneration efficacies, which makes it difficult to provide an optimized penetration capability corresponding to a condition of the skin to be treated.

A mesotherapy gun technology has been proposed to address the above-described matter. Since the mesotherapy gun technology employs an injection manner to directly inject pharmacological agents, the injection is effective but may become uneven. In addition, since the mesotherapy gun technology employs the injection manner, it is regulated by medical treatment laws, which requires using only limited pharmacological agents. Because of this, the mesotherapy gun technology may not be applied to various skin boosters.

In this regard, in recent years, a technology for improving a pharmacological agent injection effect has been disclosed in which both microneedles and a radio-frequency technology are used to form a vacuum during invasion and oscillate radio frequencies to stimulate the skin. In this state, when a positive pressure is applied, surrounding pharmacological agents are injected into invaded passages.

However, since this technology employs the radio-frequency technology, it requires a facility for oscillating radio frequencies. This complicates a structure and makes an operation and control difficult. In addition, this technology has good pharmacological agent penetration rate compared to that in the related art but a penetration depth of the pharmacological agents is not sufficient. Accordingly, a new concept of technology for efficiently delivering pharmacological agents is needed.

### DOCUMENT IN RELATED ART

### Patent Documents

Patent Document 1: Korean Patent Application Registration No. 10-0922138 (registered on Oct. 9, 2009, entitled “INSERTING APPARATUS FOR LIQUID IN HUMAN'S SKIN” by BOMTECH Co., Ltd.)
Patent Document 2: Korean Patent Application Registration No. 10-2206623 (registered on Jan. 18, 2021, entitled “NEEDLE TIP MOUNTED ON SKIN CARING DEVICE AND SKIN CARING DEVICE” by JEISYS MEDICAL Co., Ltd.)
Patent Document 3: Korean Patent Application Registration No. 10-2147856 (registered on Aug. 19, 2020, entitled “NEEDLE TIP FOR PROVIDING CURRENT, HANDPIECE AND SKIN TREATING DEVICE” by JEISYS MEDICAL Co., Ltd.)

### SUMMARY

The present disclosure was made for the purpose of solving the above matters, and the present disclosure is for the purpose of providing a microneedle therapy device and a microneedle therapy system which have an improved configuration in which fine holes are formed in the skin with microneedles, the active ingredients such as pharmacological agents are injected into the skin via the fine holes to induce production of collagens by stimulation to fibroblasts, negative and positive pressures are alternately generated, compressed air is supplied as pneumatic pressure to directly push the active ingredients so that the active ingredients are easily injected into the skin, thereby alleviating pain, achieving good active ingredient injection capability, improving a skin regenerative therapy and skin regeneration efficacy.

According to an example embodiment of the present disclosure, a microneedle therapy device equipped with a needle cover may include a pneumatic pump, a valve configured to switch a supply and cutoff of a compressed air from the pneumatic pump into the needle cover, a microneedle arranged inside the needle cover and configured to move forward and backward from an opening of the needle cover, and a controller configured to control an opening and closing of the valve and the movement of the microneedle. The controller may be configured to control an opening time of the valve based on information input by a user.

In an aspect, the controller may be configured to control power of the pneumatic pump based on the information input by the user.

In an aspect, the valve may be configured to be open while the microneedle penetrates a skin, or while the microneedle is withdrawn from the skin after reaching a final penetration depth.

In an aspect, the valve may be configured to be open for a predetermined period of time after the microneedle is completely withdrawn from the skin.

In an aspect, the information input by the user may be information about a penetration depth at which the microneedle penetrates the skin when the valve is open.

According to another example embodiment of the present disclosure, a microneedle therapy system may include the aforementioned microneedle therapy device, and a display device configured to communicate with the controller of the microneedle therapy device, configured to provide a user interface for receiving setting information about an operation of the microneedle therapy device from the user, and configured to transmit the setting information input by the user to the controller of the microneedle therapy device.

According to yet another example embodiment of the present disclosure, a method for injecting active ingredients into a skin using the aforementioned microneedle therapy device may include: setting, by the controller, an opening time of the valve, based on the information input by the user; operating the pneumatic pump to generate a pneumatic pressure in a predetermined inner space of the microneedle therapy device; and moving the microneedle arranged inside the needle cover forward and backward from the opening of the needle cover. In the act of moving the microneedle, the valve provided between the predetermined inner space and the needle cover may be configured to be open based on the opening time of the valve set by the controller so that the pneumatic pressure is applied into the needle cover.

According to the present disclosure, the following effects may be obtained.

Firstly, by supplying compressed air as pneumatic pressure into microneedles to directly push active ingredients held by the microneedles while alternately generating a negative pressure and a positive pressure with a pneumatic pump and a solenoid valve, it is possible to easily inject the active ingredients into a skin.

Secondly, by controlling one or more of an opening/closing time point of the valve and power of the pneumatic pump, it is possible to change a profile to be applied to the skin in conformity to different depths of the skin, and optimize the penetration of the active ingredients into the skin according to a condition of the skin to be treated.

Thirdly, pain is alleviated, which makes it possible to improve the usability of treatment on a patient.

### BRIEF DESCRIPTION OF THE DRAWINGS

FIG. 1 is a schematic view of a microneedle therapy system according to an example embodiment of the present disclosure.

FIG. 2 is an exemplary perspective view illustrating an appearance of a microneedle therapy device according to the present disclosure.

FIG. 3 is an exemplary cutaway cross-sectional view of a portion of the microneedle therapy device of FIG. 2.

FIG. 4 is an exemplary cross-sectional view of a microneedle unit which constitutes the microneedle therapy device according to the present disclosure.

FIGS. 5A and 5B are explanatory views illustrating a front end of a needle cover which constitutes the microneedle unit of FIG. 4, and a modified example thereof.

FIG. 6 is a schematic view illustrating an example in which active ingredients are injected using hollow microneedles constituting the microneedle unit according to the present disclosure.

FIG. 7 is an explanatory view illustrating an example in which the microneedle therapy device including the microneedles according to the present disclosure is used.

FIGS. 8A and 8B, and FIGS. 9A and 9B are explanatory views illustrating examples in which the microneedles constituting the microneedle unit according to the present disclosure are formed in a hollow structure and a solid structure, respectively.

FIG. 10 is a view illustrating an operation of a pneumatic pump and a sequence of controlling an opening/closing operation of a solenoid valve according to an example embodiment of the present disclosure.

FIG. 11A illustrates a state in which a final penetration depth is 4 mm and the microneedle according to an example embodiment of the present disclosure moves forward by 3 mm, and FIG. 11B illustrates a state in which the microneedle according to an example embodiment of the present disclosure penetrates by the final penetration depth of 4 mm and subsequently moves backward by 1 mm.

FIG. 12 is a flowchart illustrating a method for injecting active ingredients into a skin using the microneedle therapy device according to an example embodiment of the present disclosure.

### DETAILED DESCRIPTION

Hereinafter, preferred example embodiments according to the present disclosure will be described in more detail with reference to the accompanying drawings.

Before the description of the present disclosure, the following specific structure and functional descriptions are merely illustrated for the purpose of describing an example embodiment according to the concept of the present disclosure, and the example embodiments according to the concept of the present disclosure may be embodied in various forms and are not to be construed as limited to the example embodiments described herein.

In addition, the example embodiments according to the concept of the present disclosure may be varied in various forms, and specific example embodiments are illustrated in the drawings and will be described in detail. However, the example embodiments according to the concept of the present disclosure are not limited to such specific disclosures, and may include all modifications, equivalents, and alternatives falling within the spirit and scope of the present disclosure.

Further, the term “skin” used herein should be understood as the entire skin including a scalp. For example, a microneedle therapy device of the present disclosure may also be used to inject active ingredients to the scalp.

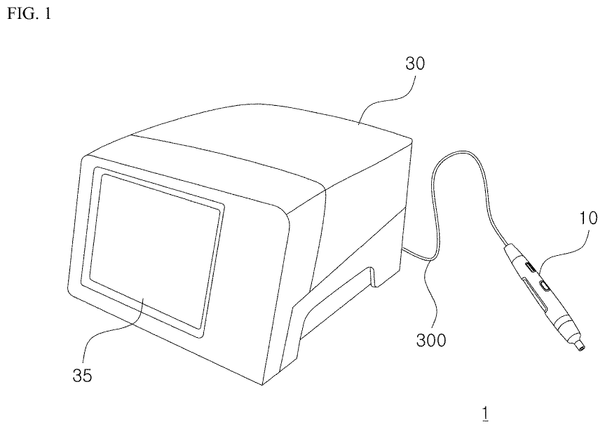

FIG. 1 is a schematic view of a microneedle therapy system 1 according to an example embodiment of the present disclosure. As illustrated in FIG. 1, the microneedle therapy system 1 may include a microneedle therapy device 10, a display device 30, and a cable 300 connecting the microneedle therapy device 10 and the display device 30.

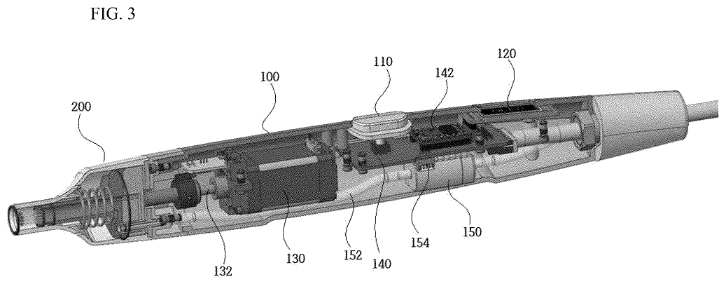

Hereinafter, the microneedle therapy system 1 will be described in detail. The display device 30 provides a user interface 35 (for example, a touch screen) to receive setting information about an operation of the microneedle therapy device 10 input by a user, and transmits the setting information input by the user to a controller 142 (see FIG. 3) of the microneedle therapy device 10 via the cable 300. The controller 142 may be a processor such as a central processing unit, a microprocessor, a multiprocessor, an integrated circuit, or equivalent thereof. Examples of the setting information may include a final penetration depth of a microneedle, a positive-pressure generation time point (a penetration depth of the microneedle when a positive pressure is generated), power of a pneumatic pump, and the like. Hereinafter, the microneedle therapy device 10 according to an example embodiment of the present disclosure will be described and subsequently, a method for controlling the microneedle therapy device 10 will be described.

### 1. Microneedle Therapy Device

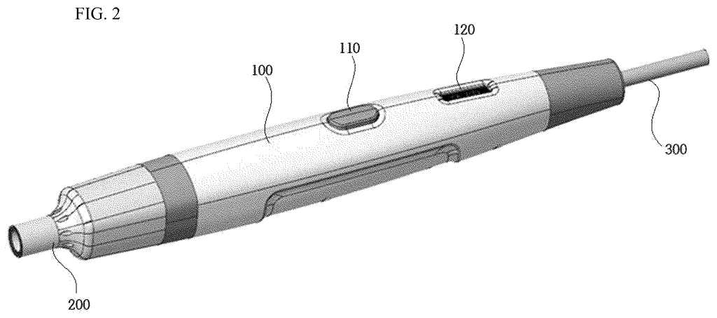

As illustrated in FIGS. 2 and 3, the microneedle therapy device 10 according to the present disclosure includes a body housing 100.

The body housing 100 defines an appearance of the microneedle therapy device 10 according to the present disclosure. In this case, a microneedle unit 200 is assembled to a front end of the body housing 100. The cable 300 is connected to a rear end of the body housing 100. An adjustment button 110 is provided in one portion of the body housing 100. A ventilation port 120 is provided in another portion of the body housing 100. Various functions such as power On-Off, the start of operation, mode switching, power adjustment, and the like may be set or adjusted by the adjustment button 110.

Further, the ventilation port 120 is a passage through which external air may be introduced into the body housing 100. A filter may be used as the ventilation port 120 if necessary.

Further, a drive power source 130 is provided inside the body housing 100. Preferably, a known linear motor may be used as the drive power source 130. In this case, unlike a typical electric motor in which a rotor rotates inside a stator, the linear motor is configured so that a mover moves linearly relative to a deployed stator at a gap along an extension line of the stator. By using the linear motor configured as above, it is possible to provide a structure in which a linear reciprocating motion rather than a rotational motion is implemented in a narrow space. A movable rod 132, which corresponds to the mover, may be provided in the drive power source 130 to be protruded therefrom. The movable rod 132 reciprocates linearly within a certain distance.

Further, a main board 140 is provided inside the body housing 100 to be spaced apart from the drive power source 130. The controller 142 is mounted on the main board 140.

In this case, the main board 140 as a printed circuit board (PCB) is a kind of microcomputer which is connected to the adjustment button 110 to provide various functions for the operation, control, setting and the like of the microneedle therapy device according to the present disclosure based on a control signal from the controller 142. To do this, the drive power source 130 is also controlled by the controller 142.

Further, a pneumatic pump 150 is provided below the main board 140 to be spaced apart from the drive power source 130. The pneumatic pump 150 is configured to compress air at a constant pressure and supply the same to the microneedle unit 200 assembled to the front end of the body housing 100. To do this, a pneumatic hose 152 is connected between the pneumatic pump 150 and the microneedle unit 200. In particular, a solenoid valve 154 may be provided in the pneumatic pump 150 to supply a certain amount of compressed air only at the time of need. In this case, the solenoid valve 154 is electrically connected to the controller 142 and an operation thereof is controlled based on a control signal from the controller 142.

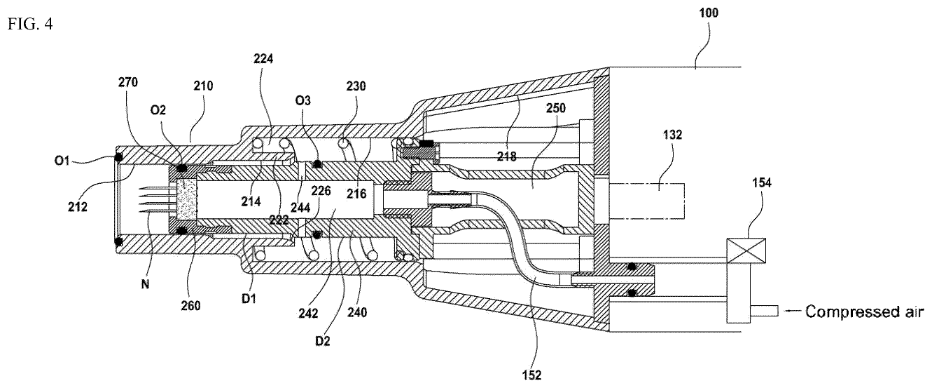

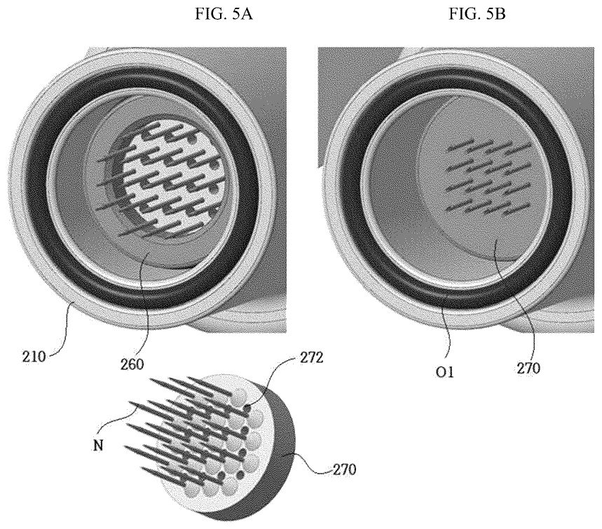

Further, as illustrated in FIG. 4 and FIGS. 5A and 5B, the microneedle unit 200 includes a needle cover 210. The needle cover 210 is assembled to the front end the body housing 100. The needle cover 210 may be formed to have a stepped structure in which a first inner-diameter portion 212, a second inner-diameter portion 214, a third inner-diameter portion 216 and a fourth inner-diameter portion 218 are sequentially formed from the front end to the rear end of the needle cover 210.

In this case, the fourth inner-diameter portion 218 assembled to the body housing 100 has the largest inner diameter, and the third inner-diameter portion 216, the second inner-diameter portion 214, and the first inner-diameter portion 212 is sequentially reduced in inner diameter. In particular, one side of the second inner-diameter portion 214 protrudes to form an inner boss 222 so that a certain size of locking groove 224 is formed between an outer circumferential surface of the inner boss 222 and the third inner-diameter portion 216.

In this case, the locking groove 224 has a substantially U shape in a cross-sectional view. An end portion of the inner boss 222, that is, an end portion of the second inner-diameter portion 214, is chamfered inward to form a tapered chamfered surface 226. The tapered chamfered surface 226 is used to increase airtightness by a third O-ring O3 (to be described later).

Further, a coil spring 230 is inserted into the third inner-diameter portion 216. A front end of the coil spring 230 is locked to the locking groove 224. In addition, a rear end of the coil spring 230 is locked to the plunger 240. Therefore, the plunger 240 is configured to be elastically pushed by the coil spring 230.

The plunger 240 is formed as a cylindrical member having an inner hollow portion 242. A rear end of the plunger 240 is flange-fixed to one end of a connection tube 250. In addition, a rear end of the connection tube 250 is connected and fixed to a front end of the movable rod 132. Therefore, when the movable rod 132 reciprocates forward and backward, the connection tube 250 also moves so that the plunger 240 reciprocates forward and backward.

In addition, the pneumatic hose 152 is provided to penetrate the connection tube 250. An end portion of the pneumatic hose 152 is connected and fixed to the hollow portion 242 of the plunger 240 such that they are in communication with each other. In this case, the pneumatic hose 152 provided to penetrate the connection tube 250 is configured to be flexible. Thus, when the connection tube 250 moves, the pneumatic hose 152 smoothly guides the motion of the connection tube 250 without being cut. Of course, the motion range of the connection tube 250 is small, so it's not matter.

In addition, an exhaust hole 244 is formed in a periphery of the plunger 240. The exhaust hole 244 is in communication with the hollow portion 242. Specifically, the exhaust hole 244 is formed outward to be spaced apart from the end portion of the inner boss 222 when the plunger 240 is located at a home position. That is, the plunger 240 has a first outer-diameter portion D1 corresponding to the first inner-diameter portion 212 and a second outer-diameter portion D2 corresponding to the second inner-diameter portion 214 which are provided sequentially from the front end to the rear end of the plunger 240. The exhaust hole 244 is formed in the second outer-diameter portion D2. A position immediately before the second outer-diameter portion D2 is inserted into the second inner-diameter portion 214 is the home position.

Therefore, at the home position, the exhaust hole 244 is positioned to be spaced apart from the tapered chamfered surface 226 without being inserted into the second inner-diameter portion 214.

Further, the third O-ring O3 is fixed to the second outer-diameter portion D2 to be spaced apart from the exhaust hole 244.

The third O-ring O3 may be fitted into and fixed to an O-ring groove (not illustrated) formed in the second outer-diameter portion D2, and may be formed to be spaced apart from the exhaust hole 244 toward the connection tube 250.

Further, as illustrated in FIGS. 4 and 5A, a cylindrical fixture 260 is fastened to the front end of the plunger 240.

In this case, a sealing plate 270 is provided inward of the fixture 260 to hermetically seal an opening of the frond end of the plunger 240.

To this end, a stepped portion may be formed in an inner circumferential surface of a front end of the fixture 260. Thus, the sealing plate 270 may be tightly locked to the stepped portion so that the sealing plate 270 may be firmly assembled and fixed to the front end of the plunger 240.

In addition, a plurality of microneedles N are fixed to the sealing plate 270. Each microneedle N has an inner hollow. A rear end of the microneedle N passes through the sealing plate 270 to be in communication with the hollow portion 242. With this configuration, air may flow into the first inner-diameter portion 212 via a sequence of the hollow portion 242 and the microneedles N.

Further, a second O-ring O2 is provided in an outer circumferential surface of the front end of the fixture 260 to maintain airtightness between the first inner-diameter portion 212 and the fixture 260. Further, a first O-ring O1 may be embedded in a front end surface of the needle cover 210 to maintain airtightness between the front end surface and the skin.

Further, a plurality of discharge holes 272 may be formed in the sealing plate 270. The discharge holes 272 may be formed to penetrate the sealing plate 270 such that they are in communication with the hollow portion 242. The discharge holes 272 may be provided to flow the air therethrough. Alternatively, as illustrated in FIG. 5B, the sealing plate 270 may be directly fixed to the front end of the plunger 240 without the fixture 260. In this case, the discharge holes 272 may be omitted and the air may flow only through the microneedles N.

### 2. Operation of Microneedle Therapy Device and Method for Using Same

An operation of the microneedle therapy device 10 configured as above according to the present disclosure and a method for using the microneedle therapy device 10 will be described later.

First, prior to the description of the operation of the microneedle therapy device 10 and the method for using the microneedle therapy device 10, the microneedle therapy device 10 according to the present disclosure will be conceptually described with a focus on obvious differences from a technology in the related art.

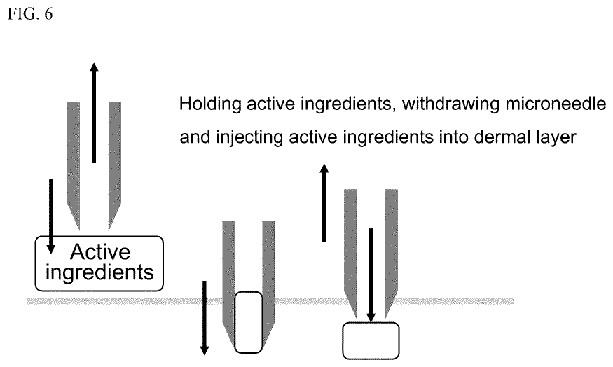

As illustrated in FIG. 6, the microneedle therapy device 10 according to an example embodiment of the present disclosure uses a method for applying active ingredients such as pharmacological agents to the skin and injecting the same into a dermal layer of the skin with the microneedles N instead of using an injection liquid. FIG. 6 illustrates an example embodiment in which hollow microneedles N are used.

As described above, the microneedle therapy device 10 according to an example embodiment of the present disclosure does not use the injection liquid. Therefore, the present disclosure is not regulated by medical treatment laws. Thus, the present disclosure may be applied to various skin boosters other than limited active ingredients.

That is, as illustrated in FIG. 6, active ingredients may be injected to the skin by applying a skin booster as active ingredients to the skin, followed by bring each microneedle N close to the skin so that the active ingredients is received in the central hollow of each microneedle N, followed by penetrating each microneedle N into the skin, followed by blowing compressed air into the central hollow of each microneedle N at the time of penetrating each microneedle N into the skin or after each microneedle N penetrates into the skin, followed by withdrawing each microneedle N from the skin.

Next, the operation of the microneedle therapy device 10 according to an example embodiment of the present disclosure and the method for using the same will be described in detail.

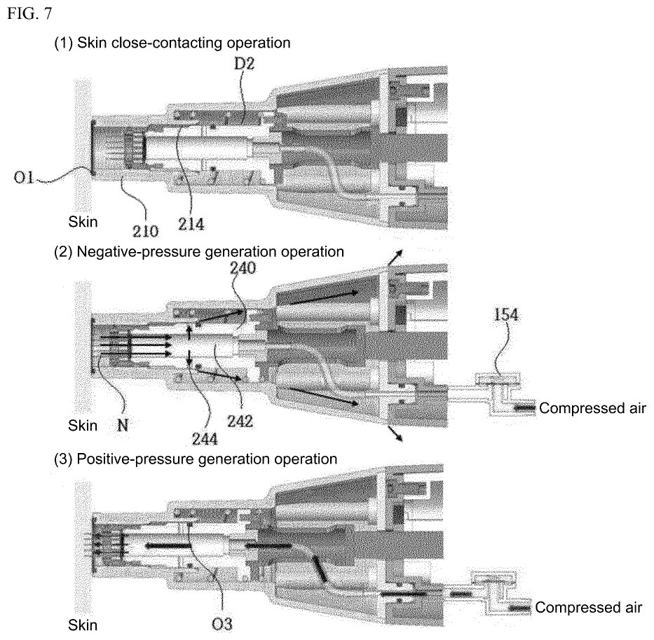

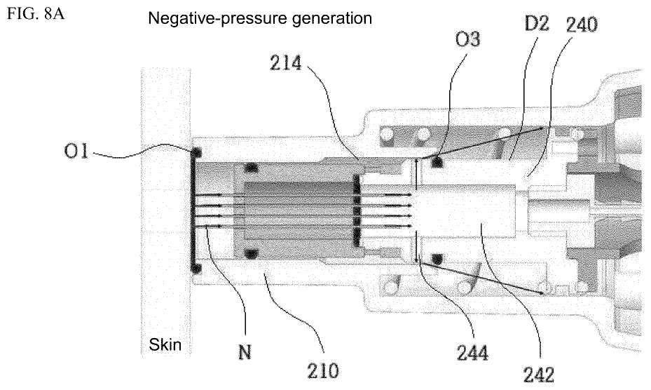

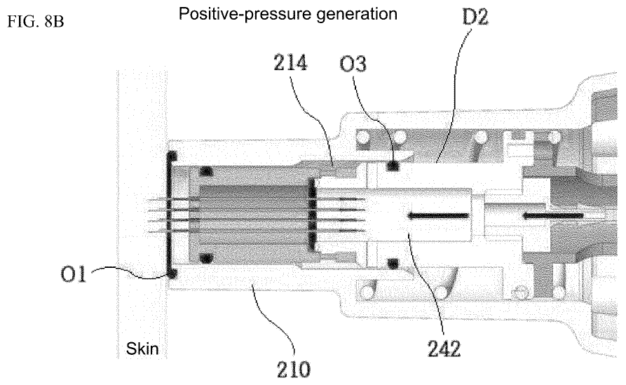

As illustrated in FIG. 7, the method for using the microneedle therapy device 10 according to an example embodiment of the present disclosure may include a skin close-contacting operation, a negative-pressure generating operation and a positive-pressure generating operation. Hereinafter, the method for using the microneedle therapy device 10 illustrated in FIG. 7 will be described with reference to an example embodiment of the microneedles N of a hollow type illustrated in FIG. 8.

First, the skin close-contacting operation is an operation of bringing the front end of the needle cover 210 of the microneedle unit 200 into close contact with the skin in a state in which active ingredients are applied onto the skin, and forming a closed space inside the needle cover 210.

According to an example embodiment of the present disclosure, the first O-ring O1 provided in the front end surface of the needle cover 210 is brought into close contact with the skin to form the closed space. In this case, the microneedles N are in a home position state. The home position state means a state in which the microneedles N are arranged to be spaced apart from the front end of the needle cover 210, and just before the second outer-diameter portion D2 is inserted inward of the second inner-diameter portion 214.

In the home position state, the pneumatic pump 150 is operated to generate the compressed air, but the supply of the compressed air is blocked by the solenoid valve 154.

The negative-pressure generating operation is an operation of discharging, inward of the third inner-diameter portion 216, air existing in the closed space between the skin and the front end of the plunger 240 via the exhaust hole 244 of the plunger 240 and the tapered chamfered surface 226 of the inner boss 222 while moving the plunger 240 forward, and forming a negative pressure in the closed space.

In this case, the air in the closed space moves to the hollow portion 242 of the plunger 240 via the inner hollow of each microneedle N and is discharged to the outside via the exhaust hole before the tapered chamfered surface 226 is sealed by the third O-ring O3.

Further, the plunger 240 is moved with the operation of the drive power source 130, which is a linear motor.

As described above, when the negative pressure is generated in the closed space, the active ingredients applied onto the skin may be easily flown into the inner hollow of each microneedle N. At this time, the supply of the compressed air remains blocked by the solenoid valve 154.

The positive-pressure generating operation is an operation of further moving the plunger 240 forward such that the microneedles N penetrate the skin while holding the active ingredients, hermetically sealing the tapered chamfered surface 226 with the third O-ring O3 and simultaneously opening the solenoid valve 154 to supply the compressed air such that the active ingredients are injected into the skin, and subsequently, moving the plunger 240 backward and withdrawing the microneedles N from the skin to further supply the compressed air for several seconds such that the closed space is kept at a positive pressure.

A sequence of operations as described above may provide the same effects as those of directly injecting active ingredients into the skin with needles. This makes it possible to inject the active ingredients into the skin in a more smooth and efficient manner.

In this case, the coil spring 230 may perform a function of applying an elastic force so that the plunger 240 may smoothly return to the home position.

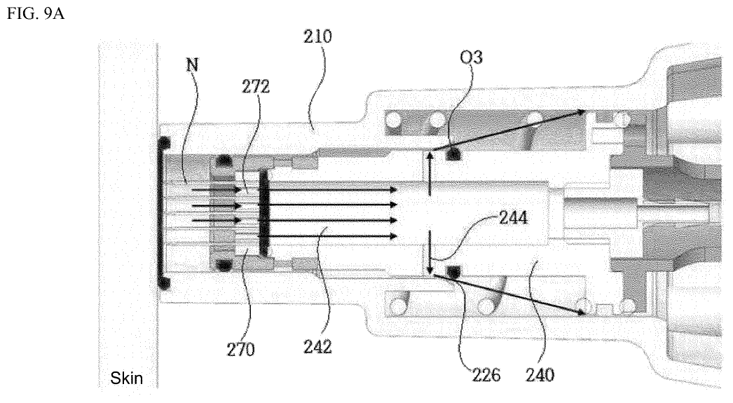

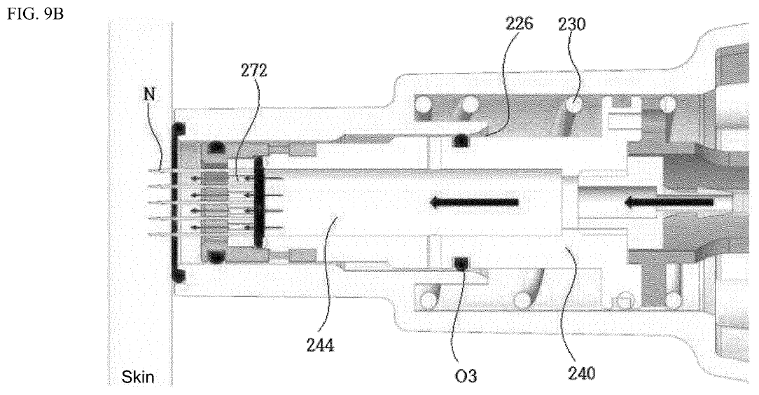

Alternatively, the microneedles N of the present disclosure may have a solid structure as illustrated in FIGS. 9A and 9B, instead of the hollow structure described above with reference to FIGS. 8A and 8B.

For example, as illustrated in the figure, the microneedles N may be configured to have a solid structure whose interior is filled. The sealing plate 270 may have a structure in which the plurality of discharge holes 272 is formed between the microneedles N penetrating through the sealing plate 270.

The microneedles N having such a solid structure may be operated as illustrated in FIGS. 9A and 9B. The operation of the microneedles N having the solid structure is substantially similar to that of the microneedles N having the hollow structure as described above, and therefore, only differences between the operation of the microneedles N having the solid structure and the operation of the microneedles N having the hollow structure will be additionally described below.

That is, when the front end of the needle cover 210 is brought into close contact with the skin in the state in which the active ingredients are applied onto the skin, the closed space is formed between the skin and the needle cover 2100, and the air existing in the closed space moves to the inner hollow portion 242 of the plunger 240 and is discharged along the tapered chamfered surface 226. As a result, the closed space is kept at the negative pressure as illustrated in FIG. 9A.

In this state, as illustrated in FIG. 9B, the plunger 240 is moved forward and the microneedles N penetrates the skin. When the third O-ring O3 is brought into close contact with the tapered chambered surface 226 and the compressed air is supplied to the inner hollow portion 242 of the plunger 240, the closed space is kept at the positive pressure so that the active ingredients applied on the skin are injected into the skin. The active ingredients may be injected via gaps between outer circumferential surfaces of the microneedles N and the skin. Thereafter, by moving the plunger 240 backward, further supplying the compressed air for several seconds while withdrawing the microneedles N, and keeping the closed space remains at the positive pressure, the active ingredients may be smoothly and efficiently injected and filled into sites from which the microneedles N are withdrawn.

A sequence of operations as described above may provide the same effects as those of directly injecting active ingredients into the skin with needles. This makes it possible to inject the active ingredients into the skin in a more smooth and efficient manner.

In this case, the coil spring 230 may perform a function of applying an elastic force so that the plunger 240 may smoothly return to the home position.

### 3. Control of Microneedle Therapy Device (Control of Positive-Pressure Generation Time Point)

As described above, in the present disclosure, the positive pressure is generated using the pneumatic pump 150 and the solenoid valve 154 rather than using a change in volume caused by a vertical movement of the plunger. This makes it possible to set a positive-pressure generation time point independently of the vertical movement of the plunger. That is, by controlling the operation of the pneumatic pump 150 and the opening/closing time point of the solenoid valve 154, it is possible to set the positive-pressure generation time point independently of the vertical movement of the plunger. Accordingly, in consideration of a condition of the skin to be treated, it is possible to optimize a profile in which the active ingredients are injected into the skin (a profile in which the active ingredients are injected to correspond to different depths of the skin). For example, the active ingredients may be injected uniformly regardless of the different depths of the skin. Alternatively, the active ingredients may be injected intensively at a specific depth. Such an action may be further maximized by adjusting power of the pneumatic pump 150.

That is, by generating and maintaining a predetermined positive pressure as soon as the microneedles N begin to penetrate the skin (or before the penetration), it is possible to uniformly inject the active ingredients regardless of the different depths of the skin. Alternatively, when the microneedles N reaches the specific depth, the compressed air of a relatively high pressure may be blown into the microneedles N to intensively inject the active ingredients to the specific depth. In the case, the specific depth may be different from that at which the microneedles N will finally penetrate the skin (hereinafter referred to as “final penetration depth”).

As described above with reference to FIGS. 1 and 3, the user may input, as pieces of setting information, a desired positive-pressure generation time point (the penetration depth of the microneedle at the time of generating the positive pressure, that is, the specific depth) and a desired power of the pneumatic pump 150, with the user interface 35 of the display device 30. The pieces of setting information are transmitted to the controller 142 of the microneedle therapy device 10. The controller 142 controls the opening/closing of the solenoid valve 154, the power of the pneumatic pump 150 and the like based on the pieces of setting information.

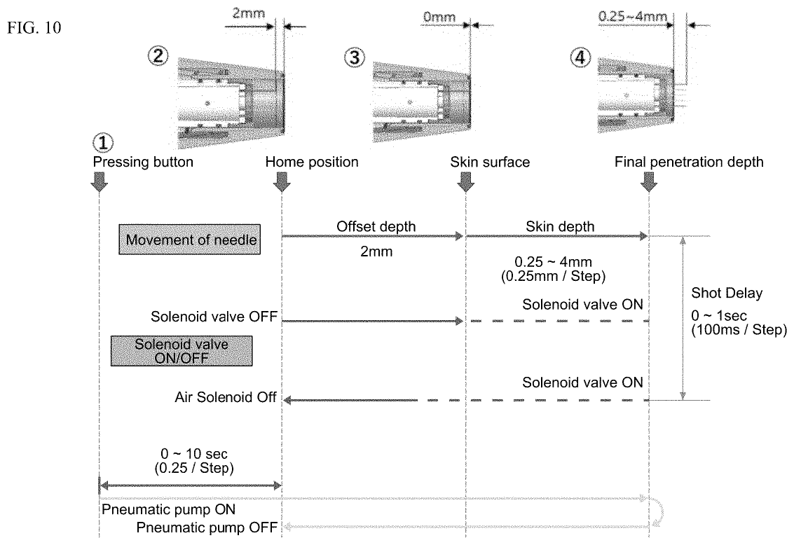

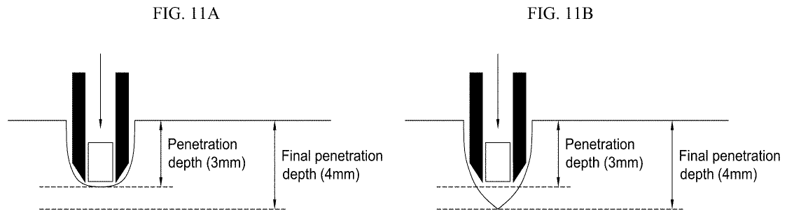

FIG. 10 illustrates a procedure of controlling the operation of the pneumatic pump 150 and the opening/closing of the solenoid valve 154 according to an example embodiment of the present disclosure.

When the operation button 110 is pressed by the user, the microneedle therapy device 10 starts to operate.

In an initial state of the microneedle therapy device 10 before the user presses the operation button 110, the pneumatic pump 150 remains turned off and the solenoid valve 154 also remains turned off (the solenoid valve 154 remains closed). When the user presses the operation button 110, the pneumatic pump 150 is turned on to generate compressed air. The flow of the compressed air is blocked by the solenoid valve 154. At this time, the microneedles N are in the home position state (for example, the microneedles N are offset inward by 2 mm from the front end of the needle cover).

When the operation button 110 is pressed by the user and a predetermined time period (about 10 seconds or less) elapses, the microneedles N starts to move forward. In the linear motor, since a movement distance of the movable rod 132 per pulse is set in advance, the movement distance of the movable rod 132, that is, a movement distance of the microneedles N may be determined based on the number of pulses.

When the microneedles N are moved by an offset distance, the microneedles N come into contact with the skin, and subsequently penetrate the skin. In the case, the offset distance may be set in a range of more than zero to 7 mm or less. The offset distance may be set according to a configuration of the microneedle unit and user's requirement. For example, when the offset distance is set in increments of 0.25 mm in the range of more than zero to 7 mm or less, specifically, in the range of more than zero to 1 mm or less, the offset distance may be set to 0.25 mm, 0.5 mm, 0.75 mm, and 1 mm. Even in the remaining range of more than 1 mm to 7 mm or less, the offset distance may be set in the same manner as the above.

In the present disclosure, the positive pressure is generated by turning (opening) the solenoid valve 154 on. That is, a time point at which the solenoid valve 154 is turned on may be the positive-pressure generation time point.

The profile in which the active ingredients are injected to correspond to different depths of the skin may vary depending on the positive-pressure generation time point, a magnitude of the positive pressure, a type of active ingredients (that is, skin booster) or the like. Further, a skin state at a site to be treated (for example, a position or state of an epidermal layer, a position or state of a dermal layer, or the like) varies depending on a condition of a person to be treated and a skin site of the person (forehead site, eye site or the like).

In the present disclosure, the user may set the positive-pressure generation time point or the magnitude of the positive pressure in consideration of the type of skin booster, a skin condition of a site to be treated, and the like to obtain a desired profile in which active ingredients are injected.

For example, when wanting active ingredients to be intensively injected at a specific depth of 3 mm in consideration of the position of the epidermal layer or the position of the dermal layer at the skin site to be treated, the user may set the positive-pressure generation time point or the magnitude of the positive pressure corresponding thereto. The specific depth of 3 mm mentioned as above is merely an example. The specific depth may be set in the range of more than zero to 7 mm or less. For example, when the specific depth is set in increments of 0.25 mm in the range of more than zero to 1 mm or less, the specific depth may be set to 0.25 mm, 0.5 mm, 0.75 mm, and 1 mm. Even in the remaining range of more than 1 mm to 7 mm or less, the specific depth may be set in the same manner as the above.

According to an example embodiment of the present disclosure, the positive-pressure generation time point may be set to correspond to a penetration depth of the microneedles N at the positive-pressure generation time point so that the user may intuitively set the positive-pressure generation time point. For example, in a case in which the user sets a depth of 3 mm as the positive-pressure generation time point (the penetration depth of the microneedles N at the positive-pressure generation time point), when the penetration depth of the microneedles N is 3 mm, the solenoid valve 154 is open to generate the positive pressure.

Further, according to the desired profile in which the active ingredients are injected, the positive-pressure generation time point may be set while the microneedles N move forward, or may be set while the microneedles N move backward.

For example, FIG. 11A illustrates a state in which, when the final penetration depth is 4 mm, the microneedle N moves forward by 3 mm. FIG. 11B illustrates a state in which, when the final penetration depth is 4 mm, the microneedle N penetrates by 4 mm, which is the final penetration depth, and subsequently move backward by 1 mm. FIGS. 11A and 11B illustrate states in which, when the penetration depth of the microneedle N is 3 mm, shapes formed in the skin by the penetration of the microneedle N are different from each other. In this case, the profile in which the active ingredients are injected may vary.

For example, as illustrated in FIG. 11B, in the case in which the positive pressure is generated while the microneedle N penetrates by the final penetration depth of 4 mm and subsequently moves backward by 1 mm, the active ingredients may be easily injected to a site from which the microneedle N is just withdrawn. In particular, the active ingredients may be intensively injected around the depth of 3 mm.

In addition, the positive pressure may be maintained for a predetermined period of time even after the microneedle N has completely withdrawn from the skin. With this configuration, even after the microneedle N has completely withdrawn from the skin, the active ingredients may be more easily injected to the site in the epidermal layer from which the microneedle N is withdrawn.

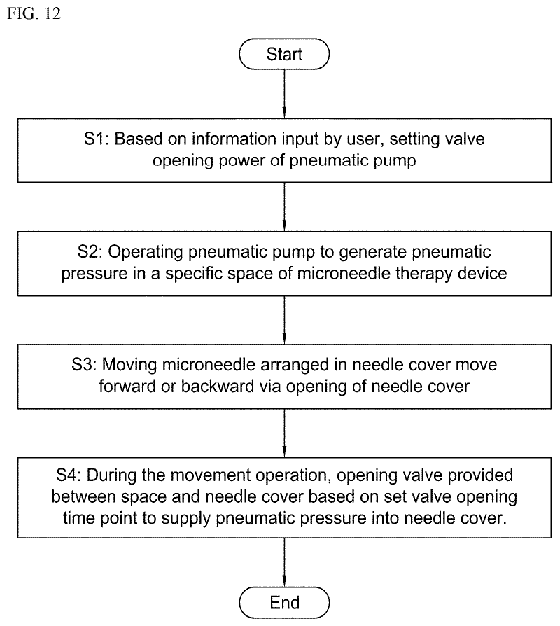

FIG. 12 is a flowchart exemplarily explaining a method for injecting active ingredients into the skin using the microneedle therapy device according to an example embodiment of the present disclosure.

As illustrated in FIG. 12, based on information input by the user (which is input using the display device and transmitted to the controller), the controller may set the valve opening time point or the power of the pneumatic pump (Operation S1). Subsequently, when the user presses the adjustment button, the pneumatic pump is operated to generate a pneumatic pressure in a specific internal space of the microneedle therapy device (Operation S2). The microneedles N arranged in the needle cover move forward or backward via the opening of the needle cover (Operation S3). During the operation of Operation S3, the solenoid valve provided between the inner space and the needle cover is open based on the set valve opening time point to supply the pneumatic pressure generated by the pneumatic pump into the needle cover (Operation S4).

### EXPLANATION OF REFERENCE NUMERALS

100: Body housing
200: Microneedle unit
300: Cable

What is claimed is:

1. A microneedle therapy device equipped with a needle cover, the microneedle therapy device comprising:
a pneumatic pump;
a valve configured to switch a supply and cutoff of a compressed air from the pneumatic pump into the needle cover;
a microneedle arranged inside the needle cover and configured to move forward and backward from an opening of the needle cover; and
a controller configured to control an opening and closing of the valve and the movement of the microneedle,
wherein the controller is configured to control an opening time of the valve based on information input by a user.

2. The microneedle therapy device of claim 1, wherein the controller is configured to control power of the pneumatic pump based on the information input by the user.

3. The microneedle therapy device of claim 1, wherein the valve is configured to be open while the microneedle penetrates a skin.

4. The microneedle therapy device of claim 1, wherein the valve is configured to be open while the microneedle is withdrawn from a skin after reaching a final penetration depth.

5. The microneedle therapy device of claim 1, wherein the valve is configured to be open for a predetermined period of time after the microneedle is completely withdrawn from a skin.

6. The microneedle therapy device of claim 1, wherein the information input by the user is information about a penetration depth at which the microneedle penetrates a skin when the valve is open.

7. A microneedle therapy system, comprising:
the microneedle therapy device of claim 1; and
a display device configured to communicate with the controller of the microneedle therapy device, configured to provide a user interface for receiving setting information about an operation of the microneedle therapy device from the user, and configured to transmit the setting information input by the user to the controller of the microneedle therapy device.

8. A method for injecting active ingredients into a skin using the microneedle therapy device of claim 1, the method comprising:
setting, by the controller, an opening time of the valve, based on the information input by the user;
operating the pneumatic pump to generate a pneumatic pressure in a predetermined space of the microneedle therapy device; and
moving the microneedle arranged inside the needle cover forward and backward from the opening of the needle cover,
wherein, in the act of moving the microneedle, the valve provided between the predetermined space and the needle cover is configured to be open based on the opening time of the valve set by the controller so that the pneumatic pressure is applied into the needle cover.

9. The method of claim 8, further comprising:
setting, by the controller, power of the pneumatic pump based on the information input by the user.

10. The method of claim 8, wherein the valve is configured to be open while the microneedle penetrates the skin.

11. The method of claim 8, wherein the valve is configured to be open while the microneedle is withdrawn from the skin after reaching a final penetration depth.

12. The method of claim 8, wherein the valve is configured to be open for a predetermined period of time after the microneedle is completely withdrawn from the skin.
